(19) United States
(12) Patent Application Publication
(10) Pub. No.: US 2026/0237200 A1
(43) Pub. Date: Aug. 13, 2026

(54) SYSTEMS AND METHODS FOR AUTOMATICALLY IDENTIFYING PHYSICAL DAMAGE IN POROUS MEDIA OBTAINED FROM A SUBSURFACE REGION

(71) Applicant: BP Corporation North America Inc., Houston, TX (US)

(72) Inventors: Danica Dralus, Houston, TX (US); Nathan Lane, Katy, TX (US); Jennie Cook, Houston, TX (US)

(73) Assignee: BP Corporation North America Inc., Houston, TX (US)

(21) Appl. No.: 19/537,165

(22) Filed: Feb. 11, 2026

Related U.S. Application Data
(60) Provisional application No. 63/757,478, filed on Feb. 12, 2025.

(51) Int. Cl. G06V 10/98; G01N 15/08; G06V 10/26; G06V 20/60

(52) U.S. Cl. CPC G06V 10/993; G01N 15/088; G06V 10/26; G06V 20/60

(57) ABSTRACT

A computer implemented method for automatically identifying physical damage in a sample of porous media obtained from a subsurface region includes receiving a digital image volume of the sample of porous media; segmenting the digital image volume into a plurality of test subvolumes; estimating, for each of the plurality of test subvolumes, one or more parameters of the test subvolume; applying one or more subvolume filters to each of the plurality of test subvolumes using the one or more estimated parameters of the test subvolumes to identify one or more of the plurality of test subvolumes as containing physical damage; filtering each of the plurality of test subvolumes identified as containing physical damage from the digital image volume to provide a filtered digital image volume of the sample of porous media; and estimating one or more petrophysical properties of the sample of porous media using the filtered digital image volume.

### CROSS-REFERENCE TO RELATED APPLICATIONS

This application claims benefit of U.S. provisional patent application No. 63/757,478 filed Feb. 12, 2025, entitled “Systems and Methods for Automatically Identifying Physical Damage in Porous Media Obtained from a Subsurface Region”, which is incorporated herein by reference in its entirety for all purposes.

### STATEMENT REGARDING FEDERALLY SPONSORED RESEARCH OR DEVELOPMENT

Not applicable.

### BACKGROUND

In various applications, it may be desired to understand how fluid flows through and/or within a porous media. Such estimates may be dependent (at least partially) on the specific properties of the porous media and the fluid(s) flowing therein. As an example, in hydrocarbon exploration and production, the porous media in question may comprise a subsurface region (e.g., an Earthen subsurface region or formation). Obtaining accurate estimates of petrophysical properties of a subsurface region may thus be important for characterizing the concentration of fluids and/or minerals within the formation. Traditionally, samples are obtained from the formation (e.g., in the form of core samples or drilling cuttings) and subjected to laboratory testing whereby petrophysical properties such as permeability, porosity, formation factor, elastic moduli, and the like of the sample may be determined. Once determined, these properties can then be used to form predictions regarding the behavior of the subsurface region which may inform a strategy for configuring well systems which may be utilized for a variety of purposes including, for example, extracting hydrocarbons or other resources from the formation, carbon capture and storage within the formation, and other applications. As an example, the determined properties may be utilized in determining the location of a planned wellbore for producing hydrocarbons from the subsurface region and/or the configuration of the planned wellbore.

### BRIEF SUMMARY OF THE DISCLOSURE

In an embodiment, a computer implemented method for automatically identifying physical damage in a sample of porous media obtained from a subsurface region comprises (a) receiving a digital image volume of the sample of porous media; (b) segmenting the digital image volume into a plurality of test subvolumes; (c) estimating, for each of the plurality of test subvolumes, one or more parameters of the test subvolume; (d) applying one or more subvolume filters to each of the plurality of test subvolumes using the one or more estimated parameters of the test subvolumes to identify one or more of the plurality of test subvolumes as containing physical damage; (e) filtering each of the plurality of test subvolumes identified as containing physical damage from the digital image volume to provide a filtered digital image volume of the sample of porous media; and (f) estimating one or more petrophysical properties of the sample of porous media using the filtered digital image volume. In some embodiments, the sample of porous media comprises a core sample obtained from the subsurface region. In certain embodiments, the one or more estimated parameters of each test subvolume comprise one or more estimated petrophysical properties of a portion of the sample of porous media corresponding the test subvolume. In other embodiments, the one or more subvolume filters comprises at least one of a morphologic filter and an outlier filter. In some embodiments, the morphologic filter applies a predefined morphologic threshold value to a morphologic score determined for each of the plurality of test subvolumes and the morphologic threshold value is based on a morphology of the physical damage. In certain embodiments, the outlier filter applies a predefined score to a standard score determined for each of the plurality of test subvolumes. In other embodiments, (d) comprises applying a plurality of the subvolume filters to each of the plurality of test subvolumes using the one or more estimated parameters of the test subvolumes to identify one or more of the plurality of test subvolumes as containing physical damage. In some embodiments, (d) comprises (d1) applying a plurality of the subvolume filters to each of the plurality of test subvolumes using the one or more estimated parameters of the test subvolumes whereby each of the plurality of subvolume filters assigns a filter score to each of the plurality of test subvolumes indicating whether or not the test subvolume contains physical damage; and (d2) identifying a test subvolume as containing physical damage in response to a proportion of the filter scores indicating the test subvolume contains damage meeting or exceeding a predefined threshold proportion. In certain embodiments, the predefined threshold proportion corresponds to a majority of the filter scores. In other embodiments, each of the plurality of test subvolumes has a size that is equal to a size of a representative elementary volume digital image volume.

In an embodiment, a system for automatically identifying physical damage in porous media obtained from a subsurface region comprises one or more processors; and a storage device coupled to the one or more processors, the storage device configured to store instructions that, when executed by the one or more processors, configure the one or more processors to receive a digital image volume of a sample of the porous media; segment the digital image volume into a plurality of test subvolumes; estimate, for each of the plurality of test subvolumes, one or more parameters of the test subvolume; apply one or more subvolume filters to each of the test subvolumes using the one or more estimated parameters of the test subvolumes to identify one or more of the test subvolumes as containing physical damage; filter each of the test subvolumes identified as containing physical damage from the digital image volume to provide a filtered digital image volume of the sample of porous media; and estimate one or more petrophysical properties of the sample of porous media using the filtered digital image volume. In some embodiments, the sample of porous media comprises a core sample obtained from the subsurface region. In certain embodiments, the one or more estimated parameters of each test subvolume comprise one or more estimated petrophysical properties of a portion of the sample of porous media corresponding the test subvolume. In other embodiments, the one or more subvolume filters comprise at least one of a morphologic filter and an outlier filter. In some embodiments, the morphologic filter is configured to apply a predefined morphologic threshold value to a morphologic score determined for each of the plurality of test subvolumes and wherein the morphologic threshold value is based on a morphology of the physical damage. In certain embodiments, the outlier filter is configured to apply a predefined score to a standard score determined for each of the plurality of test subvolumes. In other embodiments, the storage device configured to store instructions that, when executed by the one or more processors, configure the one or more processors to apply a plurality of the subvolume filters to each of the plurality of test subvolumes using the one or more estimated parameters of the test subvolumes to identify one or more of the plurality of test subvolumes as containing physical damage. In some embodiments, the storage device is configured to store instructions that, when executed by the one or more processors, configure the one or more processors to apply a plurality of the subvolume filters to each of the plurality of test subvolumes using the one or more estimated parameters of the test subvolumes whereby each of the plurality of subvolume filters assigns a filter score to each of the plurality of test subvolumes indicating whether or not the test subvolume contains physical damage; and identify a test subvolume as containing physical damage in response to a proportion of the filter scores indicating the test subvolume contains damage meeting or exceeding a predefined threshold proportion. In certain embodiments, the predefined threshold proportion corresponds to a majority of the filter scores. In other embodiments, each of the plurality of test subvolumes has a size that is equal to a size of a representative elementary volume digital image volume.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of various exemplary embodiments, reference will now be made to the accompanying drawings in which:

FIG. 1 is a block diagram of a method for obtaining a sample of porous media from a subsurface region according to some embodiments;

FIGS. 2-4 are schematic views of an exemplary system for obtaining a sample of porous media from a subsurface region according to some embodiments;

FIG. 5 is a block diagram of a method for automatically identifying physical damage in porous media obtained from a subsurface region according to some embodiments;

FIG. 6 is a schematic view of exemplary onshore and offshore sources of core samples and fluid sampling for analysis by embodiments of testing systems and methods according to some embodiments;

FIG. 7 is a schematic view of an embodiment of a testing system for analyzing core samples according to some embodiments;

FIG. 8 is a diagram illustrating an exemplary sampling strategy, according to some embodiments;

FIGS. 9A and 9B are diagrams illustrating an exemplary selection of test subvolume sizes in sampling a digital image volume according to some embodiments;

FIG. 10 is an image slice of a digital image volume segmented into a plurality of test subvolumes according to some embodiments;

FIG. 11 is a diagram illustrating an exemplary x-ray tomographic image and an exemplary graph to assess anisotropy according to some embodiments;

FIG. 12 is a block diagram of a method illustrating an exemplary predefined subvolume filter operation on test subvolumes of a sample of porous material according to some embodiments;

FIG. 13 is an image slice of a digital image volume segmented into a plurality of test subvolumes identified as containing physical damage according to some embodiments;

FIG. 14 is an image slice of a digital image volume segmented into a plurality of test subvolumes identified as not containing physical damage according to some embodiments; and

FIG. 15 is a block diagram of a computer system according to some embodiments.

### DETAILED DESCRIPTION

The following discussion is directed to various exemplary embodiments. However, one skilled in the art will understand that the examples disclosed herein have broad application, and that the discussion of any embodiment is meant only to be exemplary of that embodiment and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection, or through an indirect connection via other devices, components, and connections. In addition, as used herein, the terms “axial” and “axially” generally mean along or parallel to a central axis (e.g., central axis of a body or a port), while the terms “radial” and “radially” generally mean perpendicular to the central axis. For instance, an axial distance refers to a distance measured along or parallel to the central axis, and a radial distance means a distance measured perpendicular to the central axis.

As previously described, an accurate understanding of material flow (e.g., fluid flow) within a porous media may be dependent upon specific parameters and characteristics of the porous media and materials contained therein and forming the porous media. When the porous media comprises a subsurface region (e.g., such as in the context of hydrocarbon exploration and production), the properties and behavior of the subsurface region may be directly measured via samples obtained from the formation itself (e.g., in the form of core samples, drill cuttings, and the like). However, due to the cost and time required to directly measure petrophysical properties of formation samples of porous media (also referred to herein as “core samples” or “rock samples”), the technique of “direct numerical simulation” can be applied to efficiently predict or estimate physical properties of the subsurface region, such as porosity, absolute permeability, relative permeability, formation factor, elastic moduli, and the like of core samples, including samples from difficult rock types such as tight gas sands or carbonates.

According to this approach, a three-dimensional (3D) tomographic image volume of the core sample is obtained, for example, byway of a computer tomographic (CT) scan. The 3D image volume is divided into a plurality of distinct volume elements or “voxels” (e.g., by “thresholding” their brightness values or by another approach) to distinguish rock matrix from void space in the core sample. Direct numerical simulation of fluid flow or other physical behavior such as elasticity or electrical conductivity is then performed, from which porosity, permeability, elastic properties, electrical properties, and the like can be derived. A variety of numerical methods may be applied to solve or approximate the physical equations simulating the appropriate behavior of the core sample. These methods include, for example, lattice-Boltzmann (LB), finite element, finite difference, finite volume, numerical methods and the like.

Ultimately, as previously described, it is desirable to model the movement of fluid within a subsurface region. Accurate modeling of such behavior may be relevant for hydrocarbon production operations (e.g., determining well placement and/or configuration), and also for other considerations such as contaminant transport and rock diagenesis. The movement of fluid within a subsurface region is a function of fluid properties, and the various petrophysical properties of the rock (e.g., permeability, porosity, formation factor, elastic moduli, etc.), which may be determined as generally described above. However, issues sometimes arise in which physical damage occurs to the core sample prior to its imaging to generate an image volume thereof. For example, physical damage may occur to the core sample when it is originally obtained from the subsurface region, when it is transported from the wellsite to the laboratory for analysis, and/or when it is removed from the receptacle in which it is originally captured upon being extracted from the subsurface region. Thus, as used herein, the term “physical damage” is defined as macroscopic or structural damage that occurs to a core or other rock sample during or following its obtainment from a subsurface region.

Physical damage to the core sample may include physical fractures formed at least partially along one or more of the surfaces of the core sample, or as internal fractures not connected to external surface of the sample. Thus, this damage may be captured in the image volume of the core sample. Physical damage, given that it is not representative of the given core sample as a whole, may reduce the accuracy of the estimation of one or more petrophysical properties of the core sample using the image volume, and may impede the modeling or simulation of the subsurface region using the core sample and the image volume thereof. Therefore, in conventional practice, core samples which have incurred physical damage (with such damage happening to be manually identified by a laboratory technician and the like) may need be undesirably discarded given that the analysis thereof may be tainted by the presence of said physical damage.

Accordingly, embodiments disclosed herein include systems and methods for automatically identifying physical damage in a sample of porous media (e.g., a core or other rock sample) obtained from a subsurface region are disclosed herein. Additionally, given that physical damage is often localized and not spread evenly across the image volume, embodiments disclosed herein include segmenting an image volume of the sample of porous media into a plurality of different test subvolumes, and filtering out or otherwise excluding any of the test subvolumes identified as containing physical damage (e.g., fractures and the like) to provide a filtered image volume of the sample of porous media that excludes the test subvolumes identified as including physical damage. In this manner, in addition to identifying physical damage in the sample of porous media, embodiments disclosed herein may also automatically filter out or exclude any portions of the image volume of the sample containing physical damage to allow for further analysis of the filtered image volume (thus avoiding the need to exclude the damaged sample) without tainting the resulting analysis by the physical damage contained in the sample of porous media.

In some embodiments, one or more subvolume filters are applied to each of the plurality of test subvolumes to identify one or more of the plurality of test subvolumes as containing physical damage. In certain embodiments, a plurality of different subvolume filters are applied to each test subvolume with each filter providing a separate filter score indicating whether or not the given test subvolume contains physical damage. Physical damage may be identified as being contained in any of the test subvolumes in response to a proportion of the filter scores indicating that the given test subvolume contains physical damage meeting or exceeding a predefined threshold proportion (e.g., a majority). In this manner, individual biases or other limitations of a given subvolume filter may be balanced against other subvolume filters having different biases or limitations such that at least some of these biases may be cancelled out or otherwise mitigated.

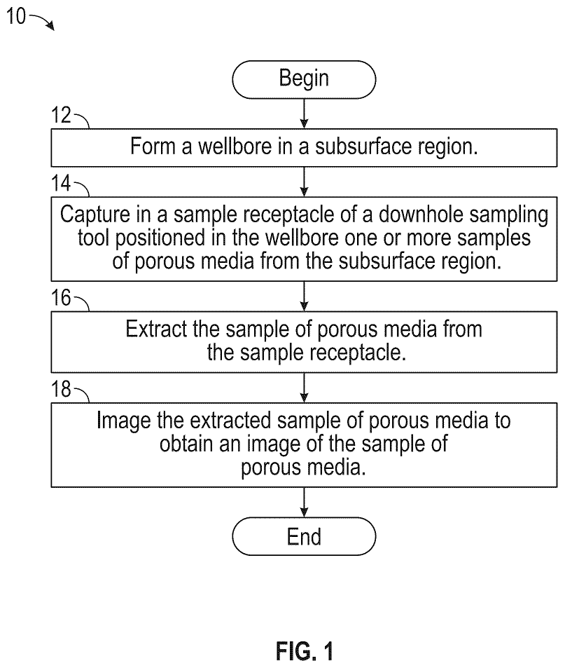

Referring now to FIG. 1, a method 10 for obtaining a sample of porous media from a subsurface region is shown. In this exemplary, the sample of porous media 1 comprises a core sample and thus may also be referred to herein simply as core sample 1. Initially, block 12 of method 10 includes forming a wellbore in a subsurface region. In some embodiments, block 12 comprises drilling by a drilling system the wellbore penetrating the subsurface region which may comprise a terranean subsurface region or formation positioned beneath a terranean surface in some embodiments.

At block 14, method 10 includes capturing in a sample receptacle of a downhole sampling tool positioned in the wellbore one or more core samples from the subsurface region. In a first example, the downhole sampling tool may physically extract samples of the subsurface region at selected surface depths which are received in one or more sample receptacles of the downhole sampling tool, such as from a sidewall of the wellbore in which the downhole sampling tool is positioned. In a second example, the samples may be acquired parallel to the axis of wellbore by a method commonly known as whole core acquisition. In some embodiments, the captured sample comprises a continuous core from the subsurface region. However, samples may be acquired using a variety of different techniques including, for instance, rotary sidewall coring, outcrop sampling, sampled from drill cuttings. In certain embodiments, the downhole sampling tool comprises a percussion sidewall coring tool as will be described further herein.

At block 16, method 10 includes extracting a core sample (or multiple core samples) from the sample receptacle (or multiple sample receptacles) of the downhole sampling tool. For example, following extraction of one or more core samples obtained from the subsurface region into one or more corresponding sample receptacles of the downhole sampling tool, the downhole sampling tool may be retrieved to the surface of the subsurface region whereby the one or more sample receptacles (each loaded with a different core sample obtained from the subsurface region) may be retrieved from the downhole sampling tool for further processing and/or analysis. In certain embodiments, at least a portion of each sample is physically extracted from its corresponding sample receptacle at block 16. The sample receptacle may remain coupled to the downhole sampling tool in some embodiments while in other embodiments the sample receptacle may first be removed from the downhole sampling tool and transported to a lab or other remote location for further processing.

At block 18, method 10 includes imaging the extracted core sample to obtain an image (e.g. a three-dimensional (3D)) image of the core sample. In certain embodiments, block 18 comprises digitally imaging the core sample with the obtained image comprising a digital image. In some embodiments, block 18 includes imaging an entirety of an outer surface of the extracted core sample while in other embodiments block 18 includes only partially imaging the outer surface of the core sample. In certain embodiments, the image obtained at block 18 of the core sample comprises a 3D tomographic image of the core sample. For instance, the 3D tomographic image may be obtained by a computer tomographic (CT) scan of the core sample. As will be discussed further herein, the 3D image may be divided into a plurality of distinct volume elements or voxels to distinguish rock matrix of the core sample from void space thereof.

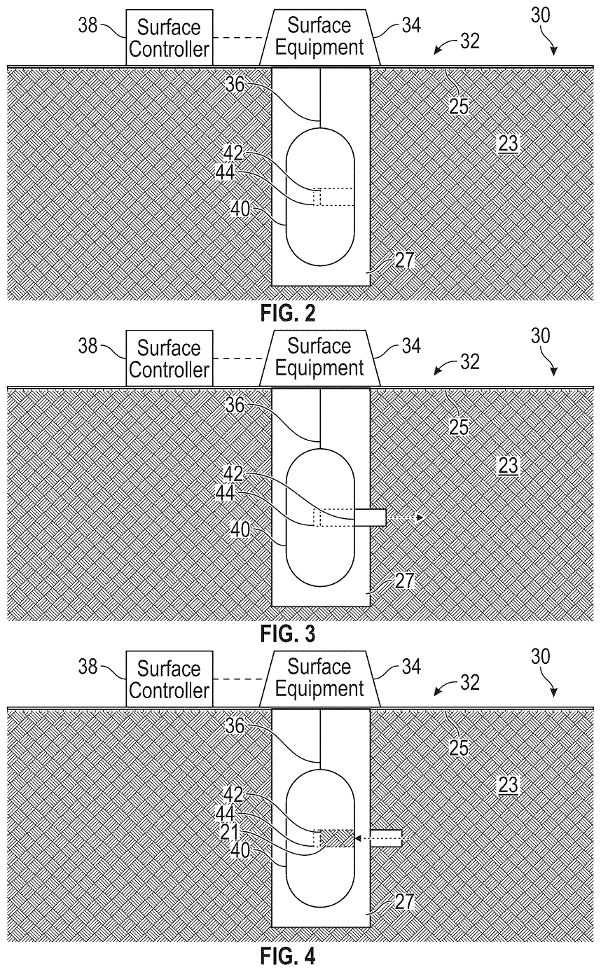

Referring to FIGS. 2-4, an exemplary embodiment of a core sampling system 30 for obtaining a sample of porous media 21 from a subsurface region 23 extending beneath a surface 25 is shown. In this exemplary, the sample of porous media 21 comprises a core sample and thus may also be referred to herein simply as core sample 21 and core sampling system 30 may similarly be referred to herein as core sampling system 30. Alternatively, porous media 21 may be obtained via techniques other than core sampling and thus may not necessarily comprise a core sample. Particularly, core sampling system 30 illustrates a porous media sampling operation being performed by a downhole sampling tool 40 suspended in a wellbore 27 by a deployment system 32, where wellbore 27 extends into the subsurface region 23 from the surface 25 thereof.

In this exemplary embodiment, deployment system 32 comprises surface equipment 34 and wireline 36 that is suspended from surface equipment 34. Particularly, wireline 36 is extendable into and retractable from the wellbore 27 via operation of surface equipment 34 such that wireline 36 may be run into wellbore 27 with the downhole sampling tool 40 coupled to (e.g., suspended) from a terminal end thereof. In other embodiments, other suitable deployment members or strings other than wireline 36 including, for example, slickline, tubing, coiled tubing, or drill pipe, may also be used depending on the specific application. Additionally, wellbore 27 may be formed with various dimensions (e.g., diameter, depth). It may also be understood that wellbore 27 is formed using a drilling system not shown in FIGS. 2-4 which may include, among other things, a support structure (e.g., a derrick, a mast) located at the surface 25, and a drilling assembly deployable into the subsurface region 23 including a drill bit for cutting into the subsurface region 23 and which is coupled to a downhole end of a drill string suspended from the surface support structure.

Further, deployment system 32 includes a surface controller 38 also located at the surface 25 and which is configured to control the operation of surface equipment 34 and/or downhole sampling tool 40. For example, surface controller 38 may be in signal communication with surface equipment 34 along with, potentially, downhole sampling tool 40 via a signal conductor (e.g., an electrical signal conductor) of wireline 36.

Downhole sampling tool 40 may be lowered via deployment system 32 to a desired surface depth in wellbore 27 whereby downhole sampling tool 40 may be operated (e.g., by surface controller 38) to physically extract or retrieve core sample 21 from the subsurface region 23 at the desired surface depth. In this exemplary embodiment, downhole sampling tool 40 includes a sample receptacle 42 and a corresponding actuator 44 for initiating the operation of sample receptacle 42. For instance, actuator 44 may comprise an energetic or percussive element configured to fire the sample receptacle 42 laterally into a sidewall 29 of the wellbore 27 at the desired surface depth as shown particularly in FIG. 3. Alternatively, actuator 44 may comprise an electromechanical actuator, a pneumatic actuator, a hydraulic actuator, and the like. Additionally, although downhole sampling tool 40 is shown in FIGS. 2-4 as including only a single sample receptacle 42, in other embodiments, downhole sampling tool 40 may include a plurality of separate sample receptacles 42 for retrieving a plurality of separate core samples 21 from the subsurface region 23.

As shown particularly in FIG. 4, the core sample 21 may be retrieved by sample receptacle 42 from the subsurface region 23. Following retrieval of core sample 21, the downhole sampling tool 40 may be returned to the surface 25 via the operation of deployment system 32 as controlled by surface controller 38. In some embodiments, sample receptacle 42 may be removed from downhole sampling tool 40 at the surface 25 and the removed sample receptacle 42 (containing core sample 21) may be transported to a separate location such as a remote lab or facility for imaging the core sample 21 following its removal from sample receptacle 42. In some embodiments, the imaging of the core sample 21 may be performed with the core sample 21 still received in the sample receptacle 42 obviating the need to extract the core sample 21 from the sample receptacle 42 prior to the performance of said imaging.

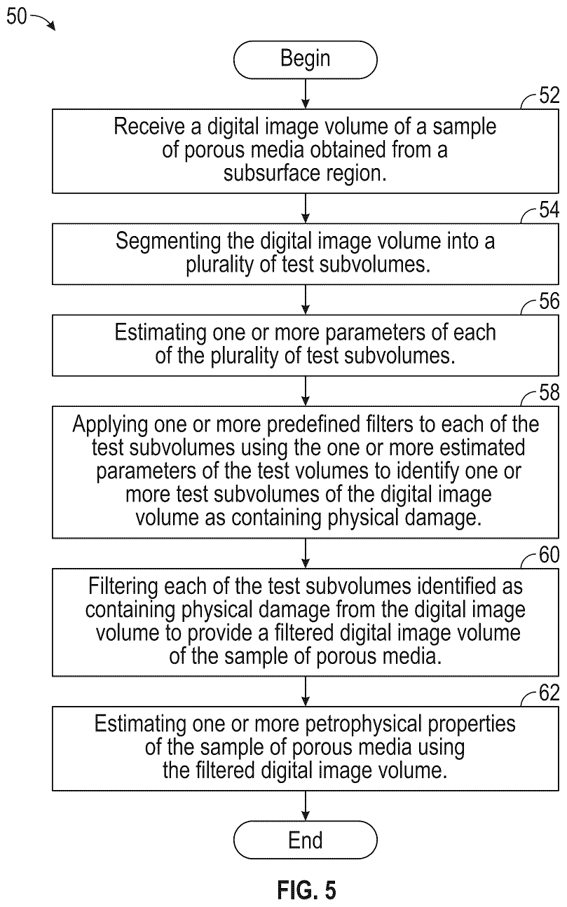

Referring to FIG. 5, an embodiment of a method 50 for automatically identifying physical damage in porous media (e.g., a core or other rock sample captured by a downhole sampling tool such as the downhole sampling tool 40 shown in FIGS. 2-4) obtained from a subsurface region (e.g., the subsurface region 23 shown in FIGS. 2-4) is shown.

Initially, at block 52, method 50 comprises receiving a digital image volume of a sample of porous media (e.g., a core sample and the like) obtained from a subsurface region. In certain embodiments, block 52 comprises receiving the digital image volume obtained at block 18 of method 10 which may, for example, comprise a digital image volume of the core sample 21 shown in FIG. 4.

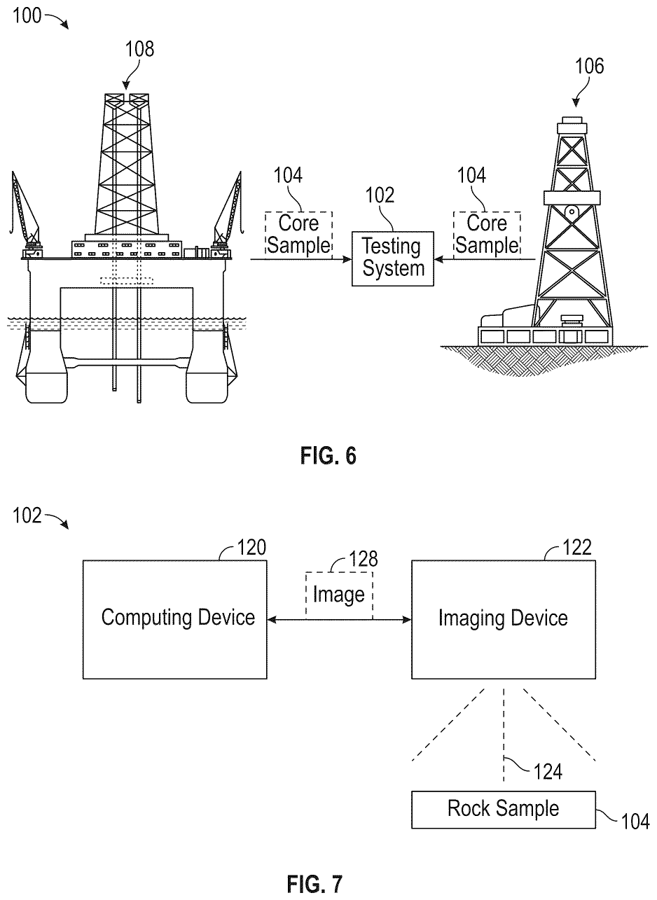

To provide an example, and referring to FIG. 6, a system 100 for acquiring and analyzing core samples 104 for purposes of generating digital image volumes of core samples (e.g., the digital image volume received at block 52 of method 50 in FIG. 5) is shown according to an exemplary embodiment. In some embodiments, system 100 may be used to analyze core samples 104 that are obtained from a subsurface region for purposes of enhancing or facilitating oil and gas production from the subsurface region. For example, in some embodiments core samples 104 can be obtained from terrestrial drilling system 106 or from marine (ocean, sea, lake, etc.) drilling system 108, either of which is utilized to extract resources such as hydrocarbons (oil, natural gas, etc.), water, and the like.

The way core samples 104 are obtained and the physical form of those samples can vary widely. Examples of core samples 104 useful in connection with embodiments disclosed herein include whole core samples, sidewall core samples (e.g., obtained from the sidewall of a wellbore extending through the subsurface region), outcrop samples, drill cuttings, and laboratory generated synthetic core samples such as sand packs and cemented packs. For instance, core samples 104 may be obtained using core sampling system 30 shown in FIGS. 2-4 in some embodiments.

A testing system 102 of system 100 is configured to acquire and analyze 3D digital image volumes 128 (shown in FIG. 7) of core samples 104 in order to determine the physical properties of the corresponding sub-surface rock. The physical properties include, for example, petrophysical properties such as those petrophysical properties that are analyzed in the context of oil and gas exploration and production.

Referring to FIG. 7, a schematic diagram of testing system 102 is shown according to some embodiments. Testing system 102 includes imaging device 122 for obtaining 2D or 3D digital image volumes, as well as other representations, of core samples 104, such digital image volumes and representations including details of the internal structure of the core samples 104. In some embodiments, imaging device 122 may be used to obtain 2D or 3D digital image volumes of a core sample 104 either when the core sample 104 is received in an at least partially surrounding sample receptacle (e.g., sample receptacle 42 shown in FIGS. 2-4) or when the core sample 104 is free of any external receptacle or structure.

An example of imaging device 122 is an X-ray CT scanner (or more simply “CT scanner”), which emits X-ray radiation 124 that interacts with an object and measures the attenuation of that X-ray radiation 124 by the object to generate a digital image volume of its interior structure and constituents. The particular type, construction, or other attributes of imaging device 122 can correspond to that of any type of X-ray device, such as a micro-CT scanner, capable of producing a digital image volume representative of the internal structure of core sample 104. Other embodiments of imaging device 122 may utilize other imaging techniques, such as, X-ray nano-tomography, focused ion beam scanning electron microscopy, nuclear magnetic resonance, etc.

In some embodiments, the digital image volume may be computationally generated rather than produced by scanning the core samples 104. In embodiments in which the digital image volume is produced by scanning a core sample 104, the core sample 104 may be a naturally occurring rock or a man-made porous material (e.g., a synthetic rock or other porous media).

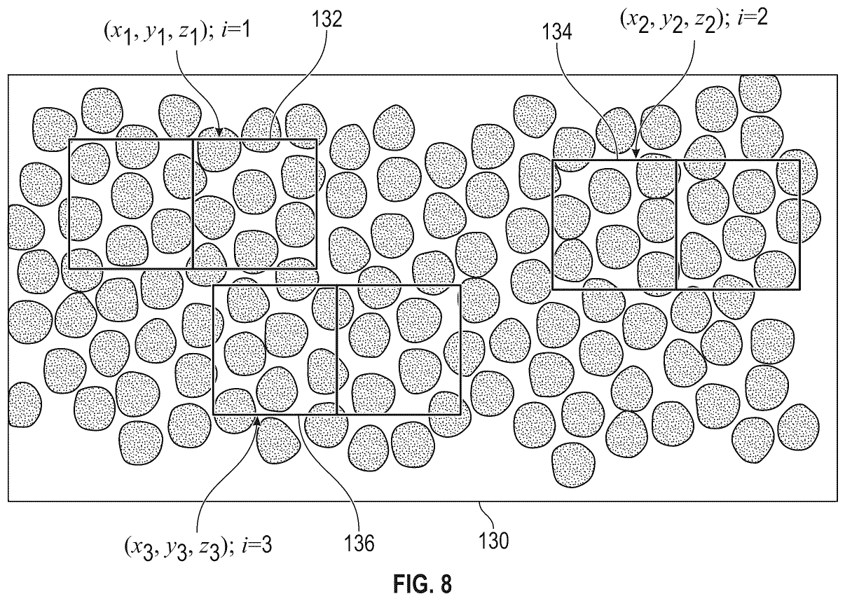

Digital image volume 128 may be composed of grayscale values representative of the attenuation of the X-ray radiation by the constituents of core sample 104. In addition, digital image volume 128 may comprise multiple two-dimensional (2D) slice images stacked along an axis of the core sample 104, which together form the digital image volume 128 of core sample 104. In general, the stacking of the 2D slice images into digital image volume 128 may be performed by computational resources of imaging device 122 itself, or by a separate computing device 120 from the series of 2D slice images produced by imaging device 122, depending on the particular architecture of testing system 102. Computing device 120 may comprise or be similar to the computer system 200 shown in FIG. 8.

The digital image volumes produced by imaging device 122 may be partitioned into 3D regular elements called volume elements, or more commonly “voxels”. For example, each voxel may be cubic, having a side of equal length in the orthogonal X, Y, and Z directions. In some embodiments, the digital image volumes produced by the imaging device 122 may be partitioned into non-cubic grid volume elements having varying geometries. The digital image volume 128 itself may contain different numbers of voxels in the X, Y, and Z directions. Each voxel within a respective 3D digital image has an associated numeric value, or amplitude, that represents the relative material properties of the imaged sample at that location of the medium represented by the digital volume. The range of these numeric values (commonly known as the grayscale range) depends on the type of digital volume, the granularity of the values, the size of the data register (e.g., 8-bit or 16-bit values), and the like. For example, 16-bit data values enable the voxels of an X-ray tomographic digital image volume to have amplitudes ranging from 0 to 65,536 with a granularity of 1. In some embodiments, testing system 102 may perform image enhancement techniques (e.g., data smoothing, noise reduction) on the digital image volume. Likewise, other components of testing system 102 (e.g., imaging device 122 itself) may alternatively perform image enhancement in whole or in part. The digital image volumes 128 captured by the imaging device 122 may comprise the digital image volumes received at block 12 of method 10.

Referring again to FIG. 5, method 50 includes, at block 54, segmenting the digital image volume into a plurality of separate test subvolumes. In some embodiments, each of the test subvolumes corresponds to a unique number of voxels of the digital image volume. The size for each of the separate test subvolumes may be predefined by a user. In certain embodiments, block 54 includes selecting a size for each of the separate test subvolumes that corresponds to an estimated representative elementary volume (REV) of the imaged core sample. However, in other embodiments, the test subvolume size may not be based or contingent on the REV of the imaged core sample.

In an example, REV refers to the volumetric extent of a core sample from which computational experiments or physical measurements may return values that are representative of the larger, or macroscopic, homogeneous rock mass. That is, the REV may be defined as the test subvolume size at which the physical parameter being computed or measured from the test subvolume is not dependent on the particular location of the test subvolume within the overall mass. Conversely, the data from computational measurements or experiments made on a computational domain or core sample of a volume smaller than the REV may not accurately represent the pore system of the rock mass macroscopically, but the physical parameter being computed or measured will vary depending on the location of the computational domain within the rock mass. As the size of the test subvolume approaches that of the REV, the computed or measured parameter will tend toward a true representative value. Additionally, computations and experiments performed on volume sizes greater than the representative volume will return values equivalent to those obtained on the volume defined as the REV (i.e., the representative value), provided that no macroscale heterogeneities are present.

In certain embodiments, in order to determine the REV of the core sample so as to select a test subvolume size corresponding to the REV, block 54 may include defining (e.g., by computing device 120 shown in FIG. 7) a set of test subvolume sizes, each test subvolume size corresponding to a unique number of voxels among the set of test subvolume sizes. In this exemplary embodiment, for each of the set of test subvolume sizes, one or more pairs of adjacent portions of the 3D digital volume having that test subvolume size may be analyzed. Additionally, in some embodiments, one of the test subvolume sizes of the set of test subvolume sizes is selected for analysis. Further, a pair of test subvolumes of a size equal to the selected test subvolume size may be acquired from the 3D digital volume and located adjacent to one another in the 3D digital volume.

In certain embodiments, one or more material properties for each of the selected adjacent test subvolumes may be determined using direct numerical simulation or other numerical or synthetic methods. For instance, these material properties may comprise physical properties of the material of the core sample that is represented by the 3D digital volume. These material properties may include physical properties of any one or more of various types including porosity, permeability, relative permeability, electrical properties, elastic properties, geometrical properties, nuclear magnetic resonance (NMR), and the like. For example, electrical properties that may be determined include such properties as formation factor, resistivity index, tortuosity factor, cementation exponent, and saturation exponent. Additionally, elastic properties that may be determined include such properties as bulk modulus, shear modulus, Young's modulus, Poisson's ratio, compressional wave velocity, and shear wave velocity. Further, other material properties that may be determined include correlation lengths, surface to volume ratio, tortuosity, chord lengths, pore throat radii, pore size, pore shape, grain size, and grain shape, and the like.

In an example, porosity can be obtained for a segmented derivative test subvolume by dividing the total number of pore space voxels by the total number of voxels contained within the test subvolume. As another example, absolute permeability may be determined using a variety of numerical methods such as finite element, finite difference or LB methods. These numerical approaches may simulate the physics of fluid flow (e.g., single phase fluid flow) to determine permeability by either directly solving/approximating the Navier-Stokes equations or recovering the Navier-Stokes equation from a discretization of the Boltzmann equation. Additionally, geometrical properties, such as correlation lengths, chord lengths, etc. can be obtained using Monte Carlo-like methods, where certain characteristics are randomly sampled throughout each adjacent test subvolume. For instance, the correlation length can be estimated by randomly sampling two points displaced at a given distance. In some embodiments, a difference value is determined between the material property values discussed above for adjacent test subvolumes within the 3D digital volume. For example, this difference value may represent the percentage or fractional difference in the material property values between those two adjacent portions of the digital image volume, at the current test subvolume size, where the test subvolume size may be user defined. For instance, in some embodiments, the test subvolume size may correspond to an estimated REV which may be determined to be a REV based on prior knowledge. In this manner, the analysis aims to assess whether the identified REV exhibits signs of damage.

Referring briefly to FIG. 8, an example of a sampling strategy for selecting a plurality of test subvolumes 132, 134, and 136 to sample an image 130 is shown. Particularly, test subvolumes 132, 134, and 136 represent cubic volume sampling the core sample represented in image 130 at random spatial locations given by (xᵢ,yᵢ,zᵢ) where i=1:n. FIG. 8 is meant to serve as an example, and strategies for selecting or sampling subvolumes of an image (e.g., image 130) may vary in other embodiments.

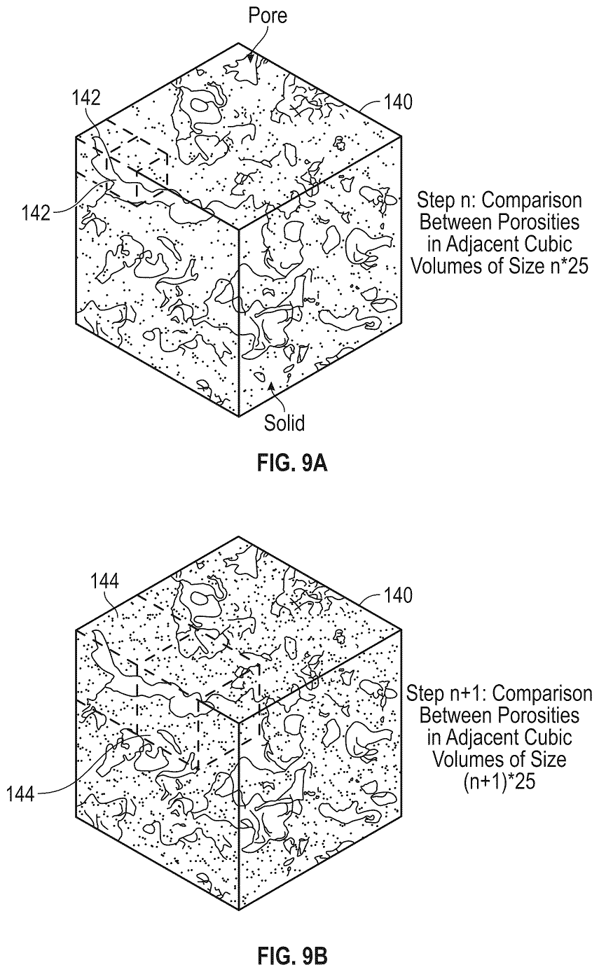

In certain embodiments, the test subvolume size may be determined iteratively. If additional test subvolume sizes are to be analyzed, the exemplary process of block 54 described above may be repeated to test an alternative subvolume size. As an example, the different test subvolume sizes may be selected in order to determine the mean difference value over multiple different sized portions of the 3D volume. For example, different test subvolume sizes may be incrementally selected to include either a greater number of voxels or fewer voxels. Referring briefly to FIGS. 9A and 9B, an exemplary 3D volume 140 is shown including a first pair of test subvolumes 142 (shown in FIG. 9A) each at a first test subvolume size, and including a second pair of test subvolumes 144 (shown in FIG. 9B) each at a second test subvolume size that is greater than the first test subvolume size. Particularly, FIGS. 9A and 9B illustrate one example of the selection of test subvolume sizes using increments of 25 voxels on a side. In this example, the first test subvolume size of test subvolumes 142 is 25 voxels on a side, while the second test subvolume size of test subvolumes 144 is 50 voxels on a side, the third test subvolume size is 75 voxels on a side (not shown in FIGS. 9A and 9B), and so on. Additionally, in this example, size refers to the length in voxels of one side of the given test subvolume 142 and 144.

Returning again to FIG. 5, in certain embodiments, upon determining that no additional test subvolume sizes remain to be analyzed, the REV for the core sample currently being analyzed may be determined. In this exemplary embodiment, by calculating the REV, an optimum (in at least some applications) size of the test subvolume may be determined that minimizes the uncertainty in the material properties simulated due to heterogeneity within the input volume. Particularly, a size of the test subvolume may be determined (corresponding to the size of the REV) that minimizes the uncertainty in the material property values without unduly increasing the size of a portion of the test subvolume to analyze. Accordingly, determining the REV when setting the size of the test subvolume may improve both computational accuracy and computational efficiency when implementing or executing method 50 on a computer system.

In some embodiments, the REV as determined for a core sample in accordance with method 50 corresponds to a volume size for which a mean difference value p (or p %) of one or more calculated material property values between two adjacent portions of a digital (test) volume of that size will differ by no more than a predetermined percentage difference value REV %. In certain embodiments, a REV may be used for subsequent direct numerical simulation measurements based on a tradeoff of a desired percentage difference value REV % between two adjacent test subvolumes on one hand, and reducing the test subvolume size on the other hand, essentially balancing the REV % with the test subvolume size.

Not intending to be bound by any particular theory, in some embodiments, the difference value p and difference value percentage p % may each be determined in accordance with Equations (1) and (2) presented below, where V_{A} and V_{B} represent material property values calculated or simulated for the adjacent test subvolumes:

$$p = 2 \times abs ( {V}_{A} - {V}_{B} ) / ( {V}_{A} + {V}_{B} ) \qquad ( 1 )$$

$$p \% = 100 \times p \qquad ( 2 )$$

As described above, the difference value p may be determined a number of different times for each test subvolume size. From the set of difference values p for each test subvolume size, the mean difference value <p> or mean difference value as a percentage p % (<p %>) may be determined. As an example, and not intending to be bound by any particular theory, the mean difference value <p> and the mean difference value percentage <p %> may be determined in accordance with Equations (3) and (4) presented below, where n represents the total number of times the difference value p or p % has been determined for each test subvolume size, and i represents the index of different value p or p % for a specific instance of two adjacent test subvolumes at that test subvolume size:

$$〈 p 〉 = \frac{1}{n} {\sum }_{i = 1}^{n} {p}_{i} \qquad ( 1 )$$

$$〈 p \% 〉 = \frac{1}{n} {\sum }_{i = 1}^{n} p {\%}_{i} \qquad ( 2 )$$

In certain embodiments, after the difference value <p> or percentage <p %> has been determined for two adjacent test subvolumes as given above, the mean difference value <p> or <p %> is determined over that newly determined value in combination with the previous determined values at that test subvolume size. The above description for determining the REV of a given image is only exemplary and may vary in other embodiments. In still other embodiments, the test subvolume size may not correspond to the REV of the image depending on the requirements of the given application.

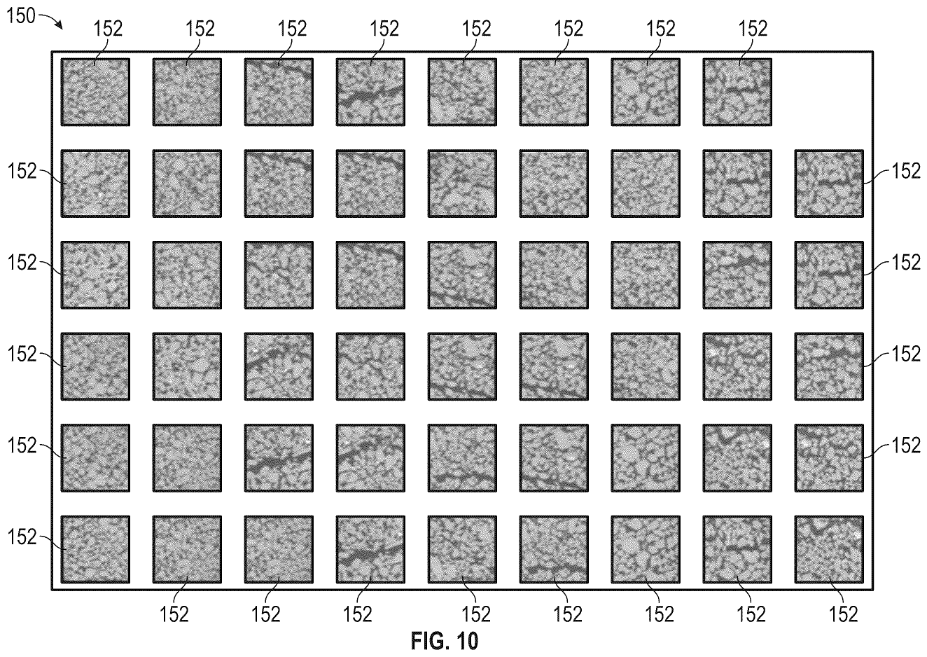

Referring briefly to FIG. 10, a slice image 150 is shown taken in the y-plane of a 3D digital image volume of a core sample. In this example, the slice image 150 has been segmented into approximately 63 test subvolumes 152 (only some of which are labeled in FIG. 10 in the interest of clarity) spread across the slice image 150. In certain embodiments, slice image 150 may be segmented into test subvolumes 152 in accordance with the techniques described above with respect to block 54 of the method 50 shown in FIG. 5. For instance, in some embodiments, the size of test subvolumes 152 may correspond to a REV of the slice image 150. Although test subvolumes 152 are shown as non-overlapping in FIG. 10, in some embodiments, the test subvolumes may overlap.

In certain embodiments, block 54 includes analyzing anisotropy within the digital image volume of the sample of porous media by conducting the REV analysis in a plurality of orthogonal directions. For example, a REV analysis may be conducted at block 54 by selecting adjacent test subvolumes aligned in the x-direction. Subsequently, in this example, the REV analysis may then be conducted by selecting adjacent test subvolumes aligned in the z-direction. Plots of the mean difference value percentage or the cumulative mean difference value percentage for each of the x- and z-directions may then be compared. If anisotropy is present within the volume, a difference in the shape of the mean (or cumulative mean) difference curves for the x- and z-directions may be evident. In other embodiments, exploring additional orientation through rotated subvolumes could provide a more comprehensive assessment, akin to principal component analysis (PCA). For instance, the adjacent subvolumes may not be aligned in the x-direction and instead may be aligned in directions that extend at an angle from the x-direction.

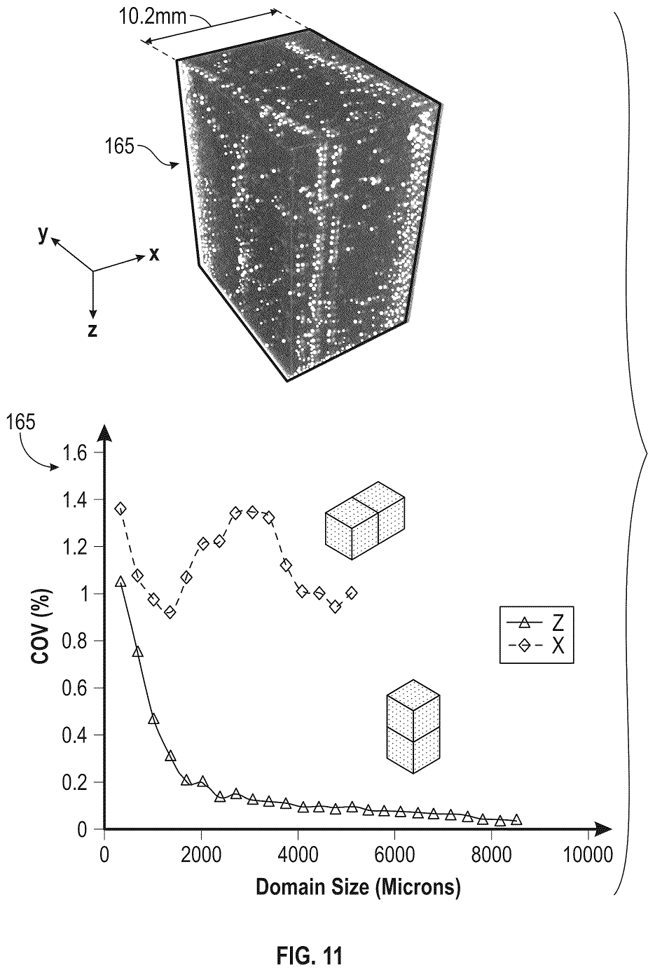

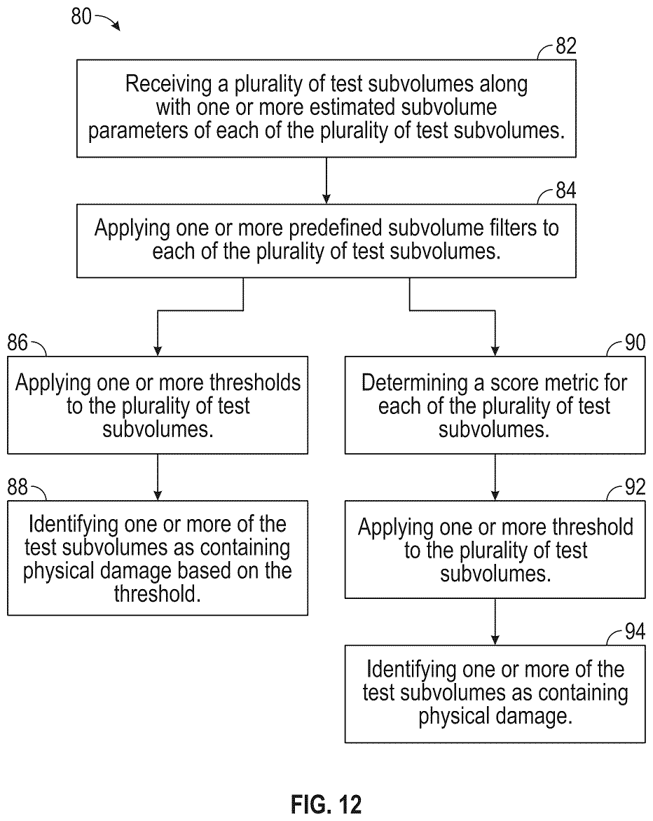

Referring briefly now to FIG. 11, an example of an x-ray tomographic digital image volume 160 along with a corresponding covariance graph 165 are illustrated to assess such anisotropy, according to an example implementation. Exemplary digital image volume 160 exhibits layering heterogeneity in the x-direction and has a resolution of 13.6 microns per voxel. Graph 165 illustrates an exemplary implementation of block 54 of the method 50 shown in FIG. 5 that assesses anisotropy, by way of a plot of coefficient of variation for probe directions along each of the x-axis and the z-axis. In this example, a covariance in grayscale values (COV) is computed, rather than a material property directly. Generally, REV analysis in this example indicates that porosity uncertainty in the z-direction decreases as volume size increases. However, porosity uncertainty in the x-direction is impacted by the heterogeneity in the core sample, which is occurring on the length scale of sedimentary layering. While the covariance drops significantly with domain size along the z-direction, covariance varies with domain size along the x-direction in response to the layering heterogeneity. Comparison of these covariance characteristics demonstrates the presence of anisotropy within the digital image volume 160 of this example.

Returning to FIG. 5, in some embodiments, block 54 assesses the REV % volume when larger scale heterogeneity is present in the digital image volume. That is, in some circumstances the desired uncertainty in terms of REV % for a certain material property can have a domain size which is greater than that of the entire digital image volume itself. In such instances, block 54 may include determining a REV % by fitting a power law to the mean difference data plot obtained from the finite digital image volume and extrapolating the result to larger domain sizes.

At block 56, method 50 includes estimating one or more parameters of each of the plurality of test subvolumes. The one or more parameters may comprise material or petrophysical properties including, for example, porosity, absolute permeability, relative permeability, formation factor, elastic moduli, and others. As described above, porosity for each test subvolume can be obtained by dividing the total number of pore space voxels contained in a given test subvolume by the total number of voxels contained within the given test subvolume. As another example, absolute permeability of each test subvolume may be determined using a variety of numerical methods such as finite element, finite difference or LB methods which simulate the physics of fluid flow to determine permeability. Further, geometrical properties of each test subvolume, such as correlation lengths, chord lengths, etc. can be obtained using Monte Carlo-like methods.

At block 58, method 50 includes applying one or more predefined subvolume filters or tests to each of the test subvolumes using the one or more estimated parameters of the test subvolumes to identify one or more test subvolumes of the digital image volume as containing physical damage. In some embodiments, the one or more predefined subvolume filters includes a morphologic filter that, based on the estimated parameters of the test subvolumes, assigns a morphologic metric to each of the test subvolumes and compares the assigned morphologic metric with a predefined value (e.g., a predefined threshold morphologic metric) to determine whether a given test subvolume contains physical damage. As used herein, a “morphologic filter” refers to image processing techniques used to selectively exclude or suppress certain characteristics of the arrangement of porous media components such as, grains and pores, while allowing, modifying or enhancing others. The morphologic metric may be based on one or more morphologic characteristic of the kinds of physical damage detected by method 50 including, for example, fractures and the like.

To provide an example, and referring briefly to FIG. 12, a method 80 illustrating an exemplary predefined subvolume filter operation on test subvolumes of a sample of porous material (e.g., core sample) is shown. In some embodiments, method 50 shown in FIG. 5 may incorporate (e.g., at blocks 58, 60, and/or 62 thereof) at least some of the features or steps of method 80; alternatively, the implementation of method 50 may vary from method 80.

As shown in FIG. 12, Method 80 begins at block 82 with receiving a plurality of test subvolumes along with one or more estimated subvolume parameters of each of the plurality of test subvolumes. The estimated subvolume parameters may include porosity, grain size, pore size, and the like. Method 80 continues at block 84 with applying one or more predefined subvolume filters to each of the plurality of test subvolumes. For example, at block 84, a morphologic filter may be applied to each of the plurality of test subvolumes. The morphologic filter may involve calculating the percentage of pore phase (e.g., porosity) or other phases present in the test subvolume. In some embodiments, block 84 includes performing one or more mathematical transformations (e.g., statistical analysis) on the one or more estimated subvolume parameters. For instance, the morphologic filter operation applied at block 84 may include performing Euclidean distance transform of the grain or pore phase.

Method 80 continues at block 86 with applying one or more predefined thresholds to the plurality of test subvolumes. In certain embodiments, the morphologic filter may employ a thresholding approach in which a threshold value of an estimated subvolume parameter is selected and the test subvolumes having an estimated subvolume parameter below the threshold value are assigned one specific numeric value, while those test subvolumes having an estimated subvolume parameter above the threshold are assigned another numeric value. For example, in some embodiments, block 86 includes applying a porosity threshold value such that test subvolumes having porosity values above the porosity threshold value are assigned a specific numeric value and test subvolumes having porosity values below the porosity threshold value are assigned another numeric value. In other embodiments, block 86 includes applying a Euclidean distance threshold and/or a grain or pore size threshold. In this approach, thresholding separates test subvolumes into groups based on the assigned numeric value to identify damaged and undamaged test subvolumes. At block 88, method 80 continues with identifying one or more of the test subvolumes as containing physical damage based on the threshold. For example, if the porosity threshold is 40%, a test subvolume with an estimated porosity value above the 40% porosity threshold may be identified as containing physical damage and test subvolumes with an estimated porosity value below 40% may be identified as undamaged.

Still referring to FIG. 12, in certain embodiments, the one or more predefined subvolume filters at block 84 include an outlier filter that, based on the estimated parameters of the test subvolume, assigns a standard score (also referred to as a “z-score”) to the test subvolume and compares the assigned standard score with a predefined value (e.g., a predefined standard score) to determine whether a given test subvolume contains physical damage. As used herein, an “outlier filter” refers to image processing techniques used to identify and exclude or suppress certain characteristics of a digital image volume. For example, at block 84, the plurality of test subvolumes may be down-sampled to reduce the image resolution by removing less important details, while retaining key features such as fractures and physical damage). Particularly, as used herein, the term “standard score” and “z-score” refers to a statistical measurement that describes a value's relationship to the mean of a group of values. The z-score is measured in terms of the number of standard deviations by which a value deviates from the mean value under consideration. In this instance, method 80 may alternatively proceed from block 84 to block 90. At block 90, method 80 continues with determining a score metric for each of the plurality of test subvolumes. For example, the z-score for each voxel in each of the plurality of test subvolumes may be determined or a single z-score may be determined for each test subvolume by considering the mean and standard deviation of its values. Method 80 continues at block 92 by applying one or more threshold to the plurality of test subvolumes.

For example, the determined z-score may compare a predefined z-score value to determine whether a given test subvolume contains physical damage. Method 80 continues at block 94 with identifying one or more of the test subvolumes as containing physical damage. For example, if a test subvolume exceeds a predefined threshold, it may be classified as damaged and if the z-score is below or within a range of z-score values, it may be classified as undamaged.

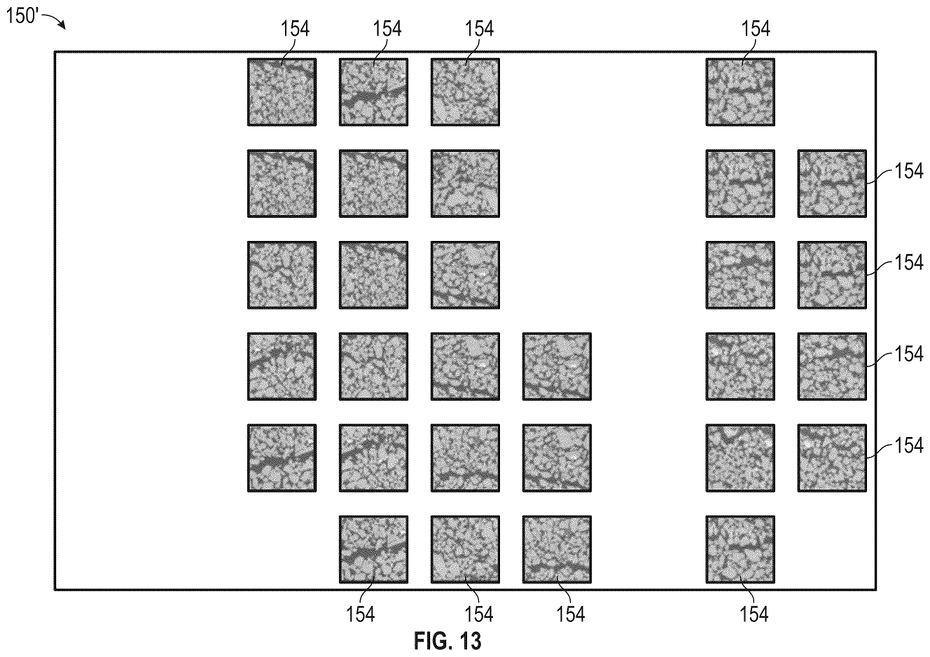

To provide a brief example, and referring briefly to FIG. 13, a version of slice image 150′ is shown with only the test subvolumes 152 of the slice image 150′ identified as containing physical damage in an exemplary implementation of the block 56 of method 50 shown in FIG. 5 being illustrated in FIG. 13. Particularly, the test subvolumes 152 of slice image 150′ identified in this example as containing physical damage are shown in FIG. 13 as damaged subvolumes 154 (only some of which are labeled in FIG. 13 in the interest of clarity).

Returning again to FIG. 5, at block 60, method 50 includes filtering, removing, or otherwise excluding each of the test subvolumes identified as containing physical damage from the digital image volume of the sample of porous media (e.g., the core sample) to provide a filtered digital image volume of the sample of porous media. In this manner, those regions of the digital image volume of the sample of porous media identified as containing physical damage may be excluded from further analysis of the sample of porous media. Particularly, given that the petrophysical properties of the test subvolumes containing physical damage may not be representative or informative of the petrophysical properties of the core sample as a whole, by excluding the test subvolumes of the digital image volume of the core sample identified as containing physical damage, said identified physical damage in the core sample may be prevented from distorting or otherwise jeopardizing the accuracy of further analysis of the filtered digital image volume such as in determining petrophysical properties of the filtered digital image volume and/or using the filtered digital image volume in the performance of modeling or simulations such as fluid flow simulations, reactive transport simulations, and the like.

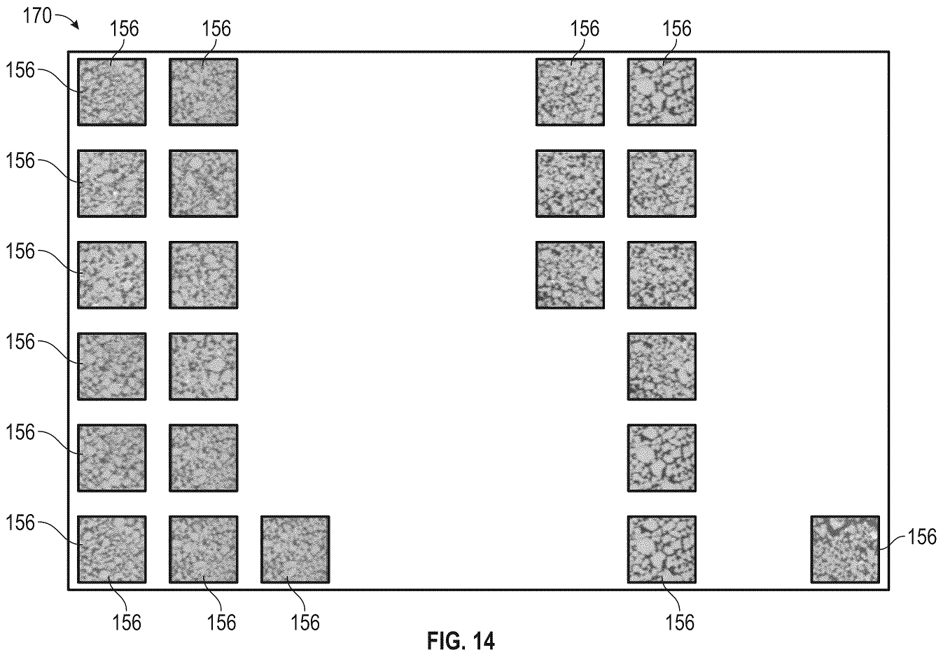

To provide a brief example, and referring briefly to FIG. 14, a filtered slice image 150 of a filtered digital image volume of the sample of porous material (e.g., the core sample) is shown. As shown in FIG. 14, filtered slice image 150 excludes the damaged subvolumes 154 indicated in the slice image 150′ of FIG. 13 and instead only includes the test subvolumes 152 of the slice image 150 that were not identified as containing physical damage in an exemplary implementation of the block 56 of method 50 shown in FIG. 5. Particularly, the test subvolumes 152 of slice image 150 identified in this example as not containing physical damage are shown in FIG. 14 as undamaged subvolumes 156 (only some of which are labeled in FIG. 14 in the interest of clarity).

In some embodiments, block 58 of method 50 comprises applying a plurality of different subvolume filters (e.g., one or more morphologic filters and/or one or more outlier filters and the like) are applied to each test subvolume with each subvolume filter providing a separate filter score indicating whether or not the given test subvolume contains physical damage. Additionally, in certain embodiments, physical damage may be identified at block 60 of method 50 as being contained in any of the test subvolumes in response to a proportion of the filter scores indicating that the given test subvolume contains physical damage meeting or exceeding a predefined threshold proportion. In certain embodiments, the predefined threshold proportion comprises a majority whereby a majority of the subvolume filters must provide a filter score indicating that a given test subvolume contains physical damage for the test subvolume to be identified at block 60 as containing physical damage. However, the threshold proportion may not necessarily correspond to a simple majority of the subvolume filters. In this manner, individual biases or other limitations of a given subvolume filter may be balanced against other subvolume filters having different biases or limitations such that at least some of these biases may be cancelled out or otherwise mitigated.

At block 62, one or more petrophysical properties of the sample of porous media (e.g., the core sample) are estimated using the filtered digital image volume of the sample of porous media. The one or more parameters may comprise material or petrophysical properties including, for example, porosity, absolute permeability, relative permeability, formation factor, elastic moduli, and others. As described above, porosity for each test subvolume can be obtained by dividing the total number of pore space voxels contained in a given test subvolume by the total number of voxels contained within the given test subvolume. As another example, absolute permeability of each test subvolume may be determined using a variety of numerical methods such as finite element, finite difference or LB methods which simulate the physics of fluid flow to determine permeability. Further, geometrical properties of each test subvolume, such as correlation lengths, chord lengths, grain and/or pore size, etc. can be obtained using Monte Carlo-like methods.

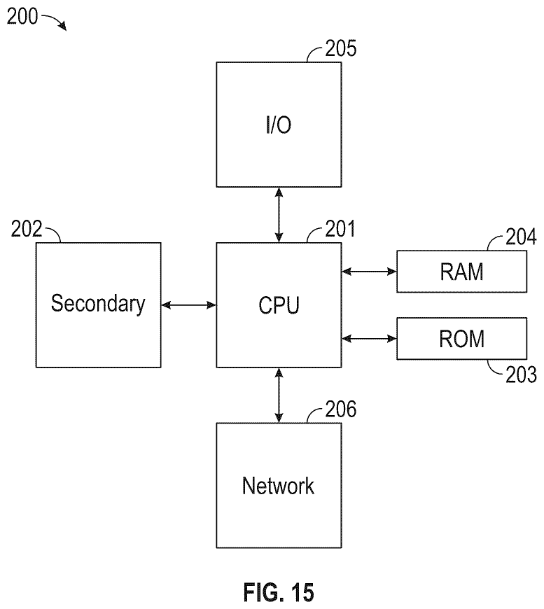

Any of the systems and methods disclosed herein can be carried out (e.g., entirely or partially) on a computer or other device comprising a processor (e.g., a desktop computer, a laptop computer, a tablet, a server, a smartphone, or some combination thereof). Referring now to FIG. 15, a computer system 200 suitable for implementing one or more embodiments disclosed herein is shown. The computer system 200 includes a processor 201 (which may be referred to as a central processor unit or CPU) that is in communication with memory devices including secondary storage 202, read only memory (ROM) 203, random access memory (RAM) 204, input/output (I/O) devices 205, and network connectivity devices 206. The processor 201 may be implemented as one or more CPU chips.

It is understood that by programming and/or loading executable instructions onto the computer system 200, at least one of the CPUs 201, the RAM 204, and the ROM 203 are changed, transforming the computer system 200 in part into a particular machine or apparatus having the novel functionality taught by the present disclosure. Thus, the RAM 204 and/or the ROM 203 may comprise a non-transitory machine-readable (or computer-readable) medium that may include instructions (which may be referred to herein as machine-readable instructions) that are executable by CPU 201 to provide functionality to computer system 200. Thus, in some embodiments, a machine-readable instructions stored on a memory may be executed on a processor, so as to configure the processor to carry out some or all of the features of the methods described herein (e.g., method 50 shown in FIG. 5).

It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware (for example in an application specific integrated circuit (ASIC), or field-programmable gate arrays (FPGA)) because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

Additionally, after the computer system 200 is turned on or booted, the CPU 201 may execute a computer program or application. For example, the CPU 201 may execute software or firmware stored in the ROM 203 or stored in the RAM 204. In some cases, on boot and/or when the application is initiated, the CPU 201 may copy the application or portions of the application from the secondary storage 202 to the RAM 204 or to memory space within the CPU 201 itself, and the CPU 201 may then execute instructions of which the application is comprised. In some cases, the CPU 201 may copy the application or portions of the application from memory accessed via the network connectivity devices 206 or via the I/O devices 205 to the RAM 204 or to memory space within the CPU 201, and the CPU 201 may then execute instructions of which the application is comprised. During execution, an application may load instructions into the CPU 201, for example load some of the instructions of the application into a cache of the CPU 201. In some contexts, an application that is executed may be said to configure the CPU 201 to do something, e.g., to configure the CPU 201 to perform the function or functions promoted by the subject application. When the CPU 201 is configured in this way by the application, the CPU 201 becomes a specific purpose computer or a specific purpose machine.

The secondary storage 202 is typically comprised of one or more disk drives or tape drives and is used for non-volatile storage of data and as an over-flow data storage device if RAM 204 is not large enough to hold all working data. Secondary storage 202 may be used to store programs which are loaded into RAM 204 when such programs are selected for execution. The ROM 203 is used to store instructions and perhaps data which are read during program execution. ROM 203 is a non-volatile memory device which typically has a small memory capacity relative to the larger memory capacity of secondary storage 202. The RAM 204 is used to store volatile data and perhaps to store instructions. Access to both ROM 203 and RAM 204 is typically faster than to secondary storage 202. The secondary storage 202, the RAM 204, and/or the ROM 203 may be referred to in some contexts as computer readable storage media and/or non-transitory computer readable media.

I/O devices 205 may include printers, video monitors, electronic displays (e.g., liquid crystal displays (LCDs), plasma displays, organic light emitting diode displays (OLED), touch sensitive displays, etc.), keyboards, keypads, switches, dials, mice, track balls, voice recognizers, card readers, paper tape readers, or other well-known input devices.

The network connectivity devices 206 may take the form of modems, modem banks, Ethernet cards, Omni-Path Architecture (OPA), InfiniBand (IB), universal serial bus (USB) interface cards, serial interfaces, token ring cards, fiber distributed data interface (FDDI) cards, wireless local area network (WLAN) cards, radio transceiver cards that promote radio communications using protocols such as code division multiple access (CDMA), global system for mobile communications (GSM), long-term evolution (LTE), worldwide interoperability for microwave access (WiMAX), near field communications (NFC), radio frequency identity (RFID), and/or other air interface protocol radio transceiver cards, and other well-known network devices. These network connectivity devices 206 may enable the processor 201 to communicate with the Internet or one or more intranets. With such a network connection, it is contemplated that the processor 201 might receive information from the network, or might output information to the network (e.g., to an event database) in the course of performing the methods (e.g., method 50) described herein. Such information, which is often represented as a sequence of instructions to be executed using processor 201, may be received from and outputted to the network, for example, in the form of a computer data signal embodied in a carrier wave.

Such information, which may include data or instructions to be executed using processor 201 for example, may be received from and outputted to the network, for example, in the form of a computer data baseband signal or signal embodied in a carrier wave. The baseband signal or signal embedded in the carrier wave, or other types of signals currently used or hereafter developed, may be generated according to several known methods. The baseband signal and/or signal embedded in the carrier wave may be referred to in some contexts as a transitory signal.

The processor 201 executes instructions, codes, computer programs, scripts which it accesses from hard disk, floppy disk, optical disk, solid state drives (SSD) (these various disk-based systems may all be considered secondary storage 202), flash drive, ROM 203, RAM 204, or the network connectivity devices 206. While only one processor 201 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 202, for example, hard drives, floppy disks, optical disks, and/or other device, the ROM 203, and/or the RAM 204 may be referred to in some contexts as non-transitory instructions and/or non-transitory information.

In an embodiment, the computer system 200 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computer system 200 to provide the functionality of a number of servers that is not directly bound to the number of computers in the computer system 200. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In an embodiment, some or all of the functionality disclosed above may be provided as a computer program product. The computer program product may comprise one or more computer readable storage medium having computer usable program code embodied therein to implement the functionality disclosed above. The computer program product may comprise data structures, executable instructions, and other computer usable program code. The computer program product may be embodied in removable computer storage media and/or non-removable computer storage media. The removable computer readable storage medium may comprise, without limitation, a paper tape, a magnetic tape, magnetic disk, an optical disk, a solid-state memory chip, for example analog magnetic tape, compact disk read only memory (CD-ROM) disks, floppy disks, jump drives, digital cards, multimedia cards, and others. The computer program product may be suitable for loading, by the computer system 200, at least portions of the contents of the computer program product to the secondary storage 202, to the ROM 203, to the RAM 204, and/or to other non-volatile memory and volatile memory of the computer system 200. The processor 201 may process the executable instructions and/or data structures in part by directly accessing the computer program product, for example by reading from a CD-ROM disk inserted into a disk drive peripheral of the computer system 200. Alternatively, the processor 201 may process the executable instructions and/or data structures by remotely accessing the computer program product, for example by downloading the executable instructions and/or data structures from a remote server through the network connectivity devices 206. The computer program product may comprise instructions that promote the loading and/or copying of data, data structures, files, and/or executable instructions to the secondary storage 202, to the ROM 203, to the RAM 204, and/or to other non-volatile memory and volatile memory of the computer system 200.

In some contexts, the secondary storage 202, the ROM 203, and the RAM 204 may be referred to as a non-transitory computer readable medium or a computer readable storage media. A dynamic RAM embodiment of the RAM 204, likewise, may be referred to as a non-transitory computer readable medium in that while the dynamic RAM receives electrical power and is operated in accordance with its design, for example during a period of time during which the computer system 200 is turned on and operational, the dynamic RAM stores information that is written to it. Similarly, the processor 201 may comprise an internal RAM, an internal ROM, a cache memory, and/or other internal non-transitory storage blocks, sections, or components that may be referred to in some contexts as non-transitory computer readable media or computer readable storage media.

The discussion above is directed to various exemplary embodiments. However, one of ordinary skill in the art will understand that the examples disclosed herein have broad application, and that the discussion of any embodiment is meant only to be exemplary of that embodiment and not intended to suggest that the scope of the disclosure, including the claims, is limited to that embodiment.

The drawing figures are not necessarily to scale. Certain features and components herein may be shown exaggerated in scale or in somewhat schematic form and some details of conventional elements may not be shown in interest of clarity and conciseness.

In the discussion above and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection of the two devices, or through an indirect connection that is established via other devices, components, nodes, and connections. In addition, when used herein (including in the claims), the words “about,” “generally,” “substantially,” “approximately,” and the like mean within a range of plus or minus 10%.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps but rather are used to simplify subsequent reference to such steps.

What is claimed is:

1. A computer implemented method for automatically identifying physical damage in a sample of porous media obtained from a subsurface region, the method comprising:
(a) receiving a digital image volume of the sample of porous media;
(b) segmenting the digital image volume into a plurality of test subvolumes;
(c) estimating, for each of the plurality of test subvolumes, one or more parameters of the test subvolume;
(d) applying one or more subvolume filters to each of the plurality of test subvolumes using the one or more estimated parameters of the test subvolumes to identify one or more of the plurality of test subvolumes as containing physical damage;
(e) filtering each of the plurality of test subvolumes identified as containing physical damage from the digital image volume to provide a filtered digital image volume of the sample of porous media; and
(f) estimating one or more petrophysical properties of the sample of porous media using the filtered digital image volume.

2. The method of claim 1, wherein the sample of porous media comprises a core sample obtained from the subsurface region.

3. The method of claim 1, wherein the one or more estimated parameters of each test subvolume comprise one or more estimated petrophysical properties of a portion of the sample of porous media corresponding the test subvolume.

4. The method of claim 1, wherein the one or more subvolume filters comprises at least one of a morphologic filter and an outlier filter.

5. The method of claim 4, wherein the morphologic filter applies a predefined morphologic threshold value to a morphologic score determined for each of the plurality of test subvolumes and wherein the morphologic threshold value is based on a morphology of the physical damage.

6. The method of claim 4, wherein the outlier filter applies a predefined score to a standard score determined for each of the plurality of test subvolumes.

7. The method of claim 1, wherein (d) comprises applying a plurality of the subvolume filters to each of the plurality of test subvolumes using the one or more estimated parameters of the test subvolumes to identify one or more of the plurality of test subvolumes as containing physical damage.

8. The method of claim 1, wherein (d) comprises:
(d1) applying a plurality of the subvolume filters to each of the plurality of test subvolumes using the one or more estimated parameters of the test subvolumes whereby each of the plurality of subvolume filters assigns a filter score to each of the plurality of test subvolumes indicating whether or not the test subvolume contains physical damage; and
(d2) identifying a test subvolume as containing physical damage in response to a proportion of the filter scores indicating the test subvolume contains damage meeting or exceeding a predefined threshold proportion.

9. The method of claim 8, wherein the predefined threshold proportion corresponds to a majority of the filter scores.

10. The method of claim 1, wherein each of the plurality of test subvolumes has a size that is equal to a size of a representative elementary volume digital image volume.

11. A system for automatically identifying physical damage in porous media obtained from a subsurface region, the system comprising:
one or more processors; and
a storage device coupled to the one or more processors, the storage device configured to store instructions that, when executed by the one or more processors, configure the one or more processors to:
receive a digital image volume of a sample of the porous media;
segment the digital image volume into a plurality of test subvolumes;
estimate, for each of the plurality of test subvolumes, one or more parameters of the test subvolume;
apply one or more subvolume filters to each of the test subvolumes using the one or more estimated parameters of the test subvolumes to identify one or more of the test subvolumes as containing physical damage;
filter each of the test subvolumes identified as containing physical damage from the digital image volume to provide a filtered digital image volume of the sample of porous media; and
estimate one or more petrophysical properties of the sample of porous media using the filtered digital image volume.

12. The system of claim 11, wherein the sample of porous media comprises a core sample obtained from the subsurface region.

13. The system of claim 11, wherein the one or more estimated parameters of each test subvolume comprise one or more estimated petrophysical properties of a portion of the sample of porous media corresponding the test subvolume.

14. The system of claim 11, wherein the one or more subvolume filters comprises at least one of a morphologic filter and an outlier filter.

15. The system of claim 14, wherein the morphologic filter is configured to apply a predefined morphologic threshold value to a morphologic score determined for each of the plurality of test subvolumes and wherein the morphologic threshold value is based on a morphology of the physical damage.

16. The system of claim 14, wherein the outlier filter is configured to apply a predefined score to a standard score determined for each of the plurality of test subvolumes.

17. The system of claim 11, wherein the storage device is configured to store instructions that, when executed by the one or more processors, configure the one or more processors to:
apply a plurality of the subvolume filters to each of the plurality of test subvolumes using the one or more estimated parameters of the test subvolumes to identify one or more of the plurality of test subvolumes as containing physical damage.

18. The system of claim 11, wherein the storage device configured to store instructions that, when executed by the one or more processors, configure the one or more processors to:
apply a plurality of the subvolume filters to each of the plurality of test subvolumes using the one or more estimated parameters of the test subvolumes whereby each of the plurality of subvolume filters assigns a filter score to each of the plurality of test subvolumes indicating whether or not the test subvolume contains physical damage; and
identify a test subvolume as containing physical damage in response to a proportion of the filter scores indicating the test subvolume contains damage meeting or exceeding a predefined threshold proportion.

19. The system of claim 18, wherein the predefined threshold proportion corresponds to a majority of the filter scores.

20. The system of claim 11, wherein each of the plurality of test subvolumes has a size that is equal to a size of a representative elementary volume digital image volume.
